(19) United States
(12) Patent Application Publication
(10) Pub. No.: US 2005/0163914 A1
(43) Pub. Date: Jul. 28, 2005

(54) Deposition method for endoprostheses provided for constantly administering medicaments

(76) Inventors: Doris Klee, Aachen (DE); Norbert Weiss, Heinsberg (DE); Alexander Rubben, Aachen (DE); Arno Bucker, Duren (DE)

Correspondence Address: Steven J Hultquist;Intellectual Property/Technology Law, P O Box 14329, Research Triangle Park, NC, 27709, US

(21) Appl. No.: 10/507,924

(22) PCT Filed: Mar. 17, 2003
(86) PCT No.: PCT/DE03/00848

(30) Foreign Application Priority Data
Mar. 16, 2002 (DE) 202 04 258.8

(51) Int. Cl. A61L 2/00; B05D 3/00

(52) U.S. Cl. 427/2.24

(57) ABSTRACT

The present invention describes a deposition method for applying an active ingredient onto an endoprosthesis with a thin polymer coating. By means of the inventive deposition method which can be carried out in a technically easy way it is possible to bring about a slow and largely constant active ingredient release, as shown in III. 1 using tretinoin as the example. Since further processing steps are not necessary after the application of the active ingredient(s), no destruction of the active ingredient by the application of a second polymer coating, for example, has to be feared. Thus, relatively unstable active ingredients, such as tretinoin, can also be applied onto the endoprosthesis without any problems.

The present invention relates to a method of producing endoprostheses (e.g. stents) having uniform active ingredient release which is due to the solubility of the active ingredient in the tissue. The stents are initially provided with a functional polymer layer to increase the active ingredient loading amount and achieve a uniform active ingredient release together with the deposition method described.

What is called the “minimally invasive methods” become more and more significant in medicine. Based on radiology, the interventional radiology should be mentioned here. It has contributed substantially to the development of minimally invasive techniques and apparatus and prostheses from suitable materials, all necessary for this purpose. For example, small metal grids are nowadays inserted in vessels as vascular endoprostheses, what is called stents, by both cardiologists and radiologists to keep said vessels open. However, conventional stents often cause the vessel walls to thicken followed by a luminal constriction in the stent area caused by cell proliferation or cell attachment.

This problem can be countered by a drug release from the stent surface which for improved drug loading and drug release can be structured and/or provided with a suitable polymeric coating. This is suggested at least by initial studies which, however, fail to contain a long-term experience over several years (Sousa J E, et al., Sirolimus-eluting stent for the treatment of in-stent restenosis: a quantitative coronary angiography and three-dimensional intravascular ultrasound study. Circulation, 2003; 107:24-27).

Various methods have been proposed for producing the coated endoprostheses. The application of a polymer coating and the subsequent binding of an active ingredient to the polymer by means of various methods can be considered prior art. In order to achieve an adequately slow active ingredient release, complicated methods have been necessary to date, in which initially the active ingredient and then a second porous polymer coating are applied onto a non-porous first polymer coating, for example, to prevent an excessively fast active ingredient release.

It is the object of this invention to further develop a method of the above mentioned type so as to permit the well-calculated build-up of a functional polymer layer which is subsequently provided with another biologically active coating, i.e. with a layer of non-covalently bonded active ingredient molecules. Here, the active ingredient release is not influenced by the polymer but depends substantially on the solubility of the active ingredient molecules in the tissue. The active ingredients to be deposited are substantially water insoluble substances (solubility of less than 0.1 mg/ml in distilled water at 25° C.) or substances poorly soluble in water (solubility of 0.1 to 0.9 mg/ml distilled water), such as e.g. tretinoin and tretinoin derivatives, orphan receptor agonists, elafin derivatives, corticosteroids and steroid hormones (such as methyl prednisolone, dexamethasone, estradiol), taxol, taxol derivatives, rapamune, tacrolimus, hydrophobic proteins or cell proliferation-altering substances. According to the invention the coating can be applied to articles having materials of different nature, such as metals, polymers, and ceramics. The endoprostheses in consideration are e.g.: stents, stent grafts, vessel clips, filters, closure systems, sheathed stents.

According to the invention the object of applying a coating with uniform active ingredient release is achieved in a method of the above mentioned type by initially producing substantially monomers in the gas phase for the production of the polymer layer from the starting compounds of general structures (1), (2) and (3) at elevated temperatures and reduced pressures, which are subsequently polymerized spontaneously by cooling, comprising 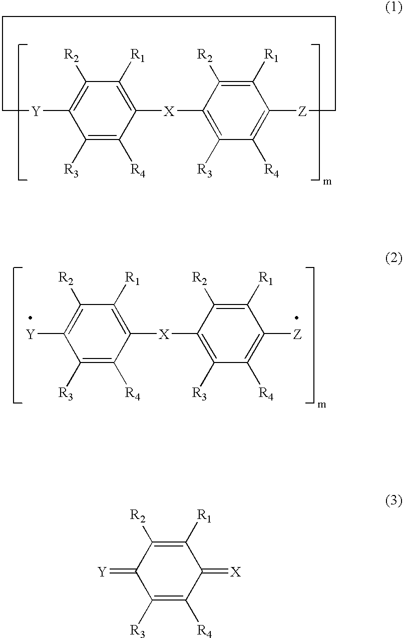
R_{1,2,3,4}: are, equal or different each, hydrogen atoms, halogen atoms, alkyl groups and/or substituted alkyl groups, aryl groups and/or substituted aryl groups, organic residues or radicals, groups of the general structure (CO(O-M-A) (wherein M: aliphatic or aromatic groups and A: e.g. hydrogen, hydroxyl, amino, carboxyl groups), metallated groups, hydroxyl groups, amino groups, carboxyl groups, ester groups, ether groups, acid halide groups, isocyanate groups, sulfur containing groups (e.g. sulfonic acid, thioether, sulfuric acid groups), nitrogen containing groups (e.g. nitrile, amide, nitro, nitrosamine groups), phosphor containing groups (e.g. phosphoric acid ester, phosphonate groups), silicon containing groups (e.g. silyl, silyloxy groups)
X, Y: hydrocarbon residues: e.g. methylene, isopropylidene, ethylene groups, functionalized hydrocarbon residues
m: number of repeating units=1-20, preferably 2-10, most preferably 2-5.

The following groups are preferred for R1-R4: hydrogen, halogen, C1-C6 alkyl, C1-C3 alkylthio, C6-C12 aryl, nitro, carbamoyl, C1-C4 alkoxy, —CN, CF₃, NH₂, carboxy, C1-C4 alkoxycarbonyl, C1-C4-N-alkyl carbamoyl or C1-C5 alkenyl.

The alkyl, alkoxy, alkylthio, alkoxycarbonyl, N-alkyl-carbamoyl, alkenyl, alkylcarboxy or alkylsulfonyl groups mentioned as residues R1, R2, R3 or R4 may be branched or unbranched and/or open-chain or cyclic. In addition, they may preferably comprise substituents, such as halogen atoms, cyano, carboxy, carbonyl, nitrile, carbamoyl, C1-C4 alkoxy, phenyl, C1-C4 alkoxycarbonyl, C1-C4 alkylcarboxy, C1-C4 N-alkylcarbamoyl, C1-C4 N-dialkylcarbamoyl, hydroxy, nitro, SO₃H, ether, sulfamoyl, C1-C4 N-alkylsulfamoyl, C1-C4 dialkylsulfamoyl, —CO—R (wherein: R═OH, O-alkyl, NH-alkyl), trifluoromethyl groupings or other open-chain or cyclic groupings having heteroatoms (e.g. with N, S and/or O), in which, where appropriate, the heteroatoms may be part of a heterocycle. The possible presence of above-mentioned substituents applies to all the groups mentioned as residues R1, R2, R3 or R4 (in so far as substitutable), in particular to the alkyl, cycloalkyl, alkenyl, cycloalkyl and C6-C12 aryl groups mentioned therein.

“Halogen” is understood to mean fluorine, chlorine, bromine or iodine.

C1-C4 alkyl is understood to mean methyl, ethyl, n-propyl, isopropyl, n-butyl, isobutyl, sec-butyl, and tert-butyl.

C1-C8 alkyl is understood to mean inter alia methyl, ethyl, n-propyl, isopropyl, n-butyl, isobutyl, sec-butyl, tert-butyl, n-pentyl, isopentyl, neopentyl, n-hexyl, 2-methylpentyl, 3-methylpentyl, 2,2-dimethylbutyl, 2,3-dimethylbutyl, n-heptyl, 2-methylhexyl, 3-methylhexyl, 2,2-dimethylpentyl, 2,3-dimethylpentyl, 2,4-dimethylpentyl, 3,3-dimethylpentyl, 3-ethylpentyl, 2,2,3-trimethylbutyl, n-octyl, 2-methylheptyl, 3-methylheptyl, 4-methylheptyl, 2,2-dimethylhexyl, 2,3-dimethylhexyl, 2,4-dimethylhexyl, 3,3-dimethylhexyl, 3,4-dimethylhexyl, 4,4-dimethylhexyl, 2-ethylhexyl, 3-ethylhexyl, 4-ethylhexyl, 2,2,3-trimethylpentyl, 2,3,3-trimethylpentyl, etc. and the cyclic equivalents thereof.

C2-C5 alkenyl is understood to mean inter alia ethene, propene, 1-butene, (cis/trans)-2-butene, 2-methylpropene, 1-pentene, 2-pentene, 2-methyl-1-butene, 3-methyl-1-butene and 2-methyl-2-butene and the cyclic equivalents thereof.

C2-C8 alkinyl is understood to mean inter alia acetylene, propyne, 1-butyne, 2-butyne, 1-pentyne, 2-pentyne, 3-methyl-I-butyne, 1-hexyne, 2-hexyne, 3-hexyne, 3,3-dimethyl-1-butyne, 1-heptyne, 2-heptyne, 3-heptyne, 1-octyne, 2-octyne, 3-octyne and 4-octyne and the cyclic equivalents thereof.

C1-C4 alkoxy is understood to mean methoxy, ethoxy, propoxy, isopropoxy, butoxy, isobutoxy, sec-butoxy and tert-butoxy.

C6-C12 aryl is understood to mean phenyl, tolyl, xylyl, naphthyl, biphenyl ring systems.

Depending on the starting compounds used, the temperatures and pressures required for the production of the monomers are between 500 and 1000° C. and less than 500 Pa, respectively.

In particular, endoprostheses having these properties can be used in human or animal vessels, vessel by-passes, vessel clips, filters, closure systems, (sheathed) stents, stent grafts, urethers, intrahepatic by-passes and in bile ducts or other hollow organs. They can be made of basic material selected from metal, metal alloys, ceramics or polymers (e.g. polyester, polyamide, polyurethane, polyethylene, polytetrafluoroethylene (PTFE)).

A concentration of functional groups increased as compared to conventional methods and usable for loading bioactive substances can be prepared in an accurately defined and adjustable proportion on the surface by the inventive method for coating endoprosthetical articles with functionalized polymers. Since side-reactions only occur to a minor extent in the mild coating method, it is possible to produce a homogeneous and well defined polymer surface. The method according to the invention also enables the well-calculated preparation of different functional groups side by side by selecting suitable monomers. This proves to be advantageous above all with respect to a simultaneous, non-covalent loading with different bioactive substances since it is thus ensured that the different functional groups can interact with the active ingredient in the most different ways. This enables a surface loading increased as compared to a simple naked metal surface or a simple polymer surface without functional groups.

Based on the invention described, polyamine-p-xylylene-co-polyxylylene is advantageously used as a polymer. It is applied onto the endoprosthesis with a layer thickness of 10 to 1000 nm, preferably 200 to 400 nm.

It has turned out that a particularly effective, functionalized polymer surface can be produced from dimers of general structure (1) or (2), wherein m=1, which are cleaved into monomers at temperatures between 600 and 900° C. and pressures of less than 150 Pa and subsequently polymerized at temperatures of less 120° C.

Bioactive water-insoluble active ingredients or active ingredients poorly soluble in water can then be deposited on the thus produced surface. In this connection, the term ‘water insoluble’ describes substances having a solubility of less than 0.1 mg/ml in distilled water at 25° C. and poorly soluble active ingredients comprise substances having a solubility of 0.1 to 0.9 mg/ml distilled water. The substances in consideration are here: tretinoin and tetrinoin derivatives, orphan receptor agonists, elafin derivatives, corticosteroids and steroid hormones (such as methylprednisolone, dexamethasone, estradiol), taxol, taxol derivatives, rapamune, tacrolimus, hydrophobic proteins or cell proliferation-altering substances or other cell proliferation-altering substances which are not soluble or are poorly soluble in water. The polymer-coated endoprosthesis is initially wet with an active ingredient solution in a water miscible solvent, such as dimethylsulfoxide (DMSO), dioxane, dimethylformamide (DMF) or tetrahydrofuran (THF) by immersion, spraying or pipetting. Thereafter, the endoprosthesis is immersed in water, the water immiscible active ingredient precipitating, partially depositing on the surface and partially incorporating into the polymer layer. The solvent is removed from the endoprosthesis during the deposition step. Based on the method described both an individual active ingredient and a combination of active ingredients can be deposited on the endoprosthesis. The deposition method of the invention differs from other substance coatings, on the one hand, by the result of the slow and uniform active ingredient release. On the other hand, methods formerly described in connection with coated endoprostheses are spray, immersion, pipetting and air flow methods and the mixing of the substance with the polymer substance to be applied (WO 00/32255, WO 00/62830, WO 98/35784). While in these methods the duration of the step carried out is decisive for the extent of active ingredient loading, the applied active ingredient amount of the proposed deposition method depends on the concentration of the active ingredient in the solution. Also, it was formerly proposed to mix the active ingredient with a polymer and subsequently release it by polymer degradation (WO 99/21908). The difficulty of applying hydrophobic substances was solved in another case by the formation of micelles which were then applied again (WO 02/085337). Another kind of substance coating describes the use of negatively charged therapeutic active ingredients and cationic polyelectrolytes (WO 01/49338). The plurality of different methods and the technical complexity of the respective methods show the difficulties of developing a technically simple and reliable drug release method. The deposition method described on the basis of this patent differs from all of the methods described thus far.

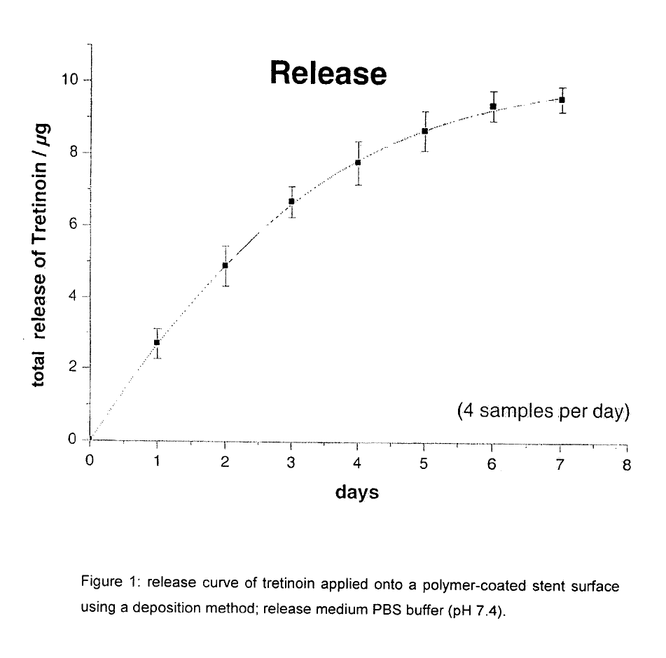

The application of a polymer onto an endoprosthesis corresponds to the current state of knowledge (DE 196 04 173 A1). Different methods of applying an active ingredient onto a polymer layer have also been previously described. Here, one of the major difficulties consists of the slow active ingredient release. It was tried to solve this slow release by the application of a second porous polymer coating onto the first one or by the influence of the first polymer coating on the active ingredient release (WO 00/32255, WO 98/36784). In the case of the present invention, only one polymer coating has to be applied and the active ingredient release curve is not influenced by the polymer either, as is the case in other drug eluting stents, but largely depends on the solubility of the active ingredient molecules in the tissue. Unlike other drug release systems, this serves for achieving an almost constant active ingredient release curve. As a result, a uniform and long-lasting effect is obtained (FIG. 1). Another advantage of the described method is that the active ingredient deposition need not necessarily be followed by the application of further coatings. The lacking destruction of the active ingredient during the application of a second polymer layer has been described to be a special problem (WO 98/35784).

In the case of the deposition method described, a minimum active ingredient residue is additionally incorporated into the polymer layer and achieves a long-term effect.

Moreover, there is the possibility of applying another polymer layer onto the functional polymer coating. The linkage can be brought about by means of spacer systems, such as diisocyanates, dicarboxylic acid chlorides or disuccinimidyl esters or by using activating coupling reagents, such as EDC or DDC. Here, a spacer system is understood to mean a molecule which is suited for a chemical linkage between the functional polymer surface and the polymer to be applied. The spacer is linked by means of functional groups, e.g. hydroxy, amino or carboxyl groups of the polymer surface. Activating coupling reagents are understood to mean substances which produce a direct chemical linkage of the polymer to be applied to the functional groups of the polymer coated surface.

The second polymer bonded to the functional polymer coating can be used for loading with an active ingredient which can be released in vivo to its environment. For the purpose of loading it is possible to use e.g. hydrogels which change their configuration as a function of temperature. At low temperatures (below body temperature), the polymer has an open structure into which the active ingredient can be introduced in a dissolved form (loading). When the temperature is raised, the polymer is closed, the active ingredient remains enclosed on the endoprosthesis surface such that it is released to its environment in a delayed fashion over a prolonged period of time.

The outer side of the stent and optionally also the inner side thereof may have a surface structure or shaping to improve the adhesion and enlarge the surface area. This structure may consist of small recesses or shapings which improve the adhesion and/or adsorptivity of the active ingredients. The outer side of the stent can be structured mechanically, thermally or chemically. The inner side can be shaped in the same way, thus creating on the outer side of the stent small recesses, for example, which have a depth of 5-50 μm and a width of 5-50 μm.

The following methods are suitable for applying one or more of the above-mentioned active ingredients onto the stent surface:

The active ingredient is deposited on a smooth and/or structured stent surface which is functionally coated with a polymer—such as polyamino-p-xylylene-co-polyxylylene.

Introduction of the active ingredient(s) into a second polymer layer which is covalently bonded to the smooth and/or structured stent surface which is functionally coated with polymer.

The invention is further described by means of the attached figures:

FIG. 1 describes the release curve of tetrinoin which was applied onto a polymer-coated stent surface using deposition methods; release medium PBS buffer (pH 7.4).

The following examples serve for further explaining the invention:

### EXAMPLE 1

In order to coat an endoprosthesis, the dimer 4-amino-[2,2]-paracyclophane is cleaved into reactive monomers at 700° C. and 20 Pa and subsequently polymerizes on the stent surface cooled to about 20° C. The aspired thickness of the polymer coating is advantageously 10 to 1000 nm, more preferably 200 to 400 nm. The subsequent non-covalent biological coating of the surface is carried out using tretinoin or tretinoin derivatives. The polymer-coated stent wet with a solution of the active ingredient in dimethylsulfoxide (DMSO) is immersed in water, the water insoluble active ingredient precipitating, partially depositing on the surface and partially incorporating into the polymer layer.

### EXAMPLE 2

In order to coat a stent, the dimer 4-amino-[2,2]-paracyclophane is cleaved into reactive monomers at 700° C. and 20 Pa and subsequently polymerizes on the stent surface cooled to about 20° C. The aspired thickness of the polymer coating is advantageously 10 to 1000 nm, more preferably 200 to 400 nm. A second polymer layer is applied by a direct covalent linkage or a spacer system. On account of its open structure at low temperatures (<36° C.), this layer can take up active ingredient molecules. At elevated temperatures ≧36° C., it has a closed structure which encloses the active ingredient molecules. The stent thus equipped with the second polymer is incubated at <36° C. with an active ingredient solution for the purpose of active ingredient loading and raised to a temperature ≧36° C. in the medium of the active ingredient solution. In this way, active ingredient molecules are enclosed on the stent surface.

1. A method of producing bioactive surfaces on an endoprosthesis wherein the endoprosthesis comprises a functional polymer layer and an active ingredient-containing layer positioned on the functional polymer layer, the method comprising:
(a) depositing starting compounds of general structures (1), (2) or (3) or a combination thereof: 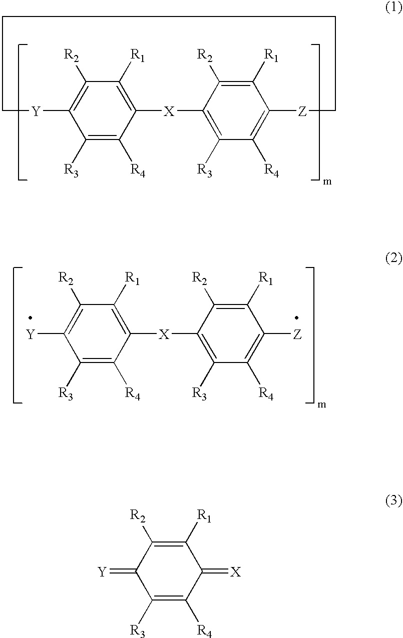
wherein R_{1,2,3,4}: are, equal or different and comprising, hydrogen atoms, halogen atoms, alkyl groups, substituted alkyl groups, aryl groups, substituted aryl groups, organic residues, organic radicals, a compound of the general structure CO (O-M-A), wherein M: is an aliphatic or aromatic groups;
A is a hydrogen, hydroxyl group, amino group carboxyl group, metallated groups, ester group, ether group, acid halide group, isocyanate group, sulfur containing group, nitrogen containing group, phosphorus containing group, or silicon containing groups (e.g. silyl, silyloxy groups)
X, Y is a hydrocarbon residue
m: number of repeating units=1-20,
at temperatures from about 500 to about 1000° C. and pressures less than 500 Pa to generate monomers in a gas phase; and
reducing the temperature to polymerize the functional polymer layer and form a polymer-coated endoprosthesis.

2. The method according to claim 1, wherein dimers of structure (1) or (2), wherein n=1, are cleaved into monomers at temperatures between 600 and 900° C. and pressures of less than 100 Pa and the subsequent polymerization is carried out at temperatures of less than 120° C.

3. The method according to claim 1, wherein the functional polymer layer deposited on the endoprosthesis has a layer thickness between 10 and 1000 nm.

4. The method according to claim 1, further comprising
wetting the polymer-coated endoprosthesis in a water misicible solvent to form a wetted polymer-coated endoprosthesis;
immersing the wetted polymer-coated endoprosthesis in water;
adding the active ingredient to the water wherein the active ingredient is poorly soluble in water and precipitates to at least partially deposits on functional polymer layer.

5. The method according to claim 4, wherein the functional polymer layer increases adhesion of the active ingredient loading compared to the non-coated surface by hydrophobic and electrostatic interactions with the functional groups of the functional polymer coating.

6. The method according to claim 4, characterized in that the active ingredient(s) are additionally incorporated in part into the polymer layer.

7. The method according to claim 4, wherein the active ingredients that is poorly soluble in water, comprises tretinoin, tretinoin derivatives, orphan receptor agonists, elafin derivatives, corticosteroids, steroidal hormones, taxol, taxol derivatives, rapamune, tacrolimus, hydrophobic proteins or cell proliferation-altering substances.

8. The method according to claims 4, wherein the kinetics of the active ingredient release in vivo from the endoprosthesis surface is determined by the poor solubility of the active ingredient in aqueous media.

9. The method according to claim 4, wherein the active ingredient-containing layer is another polymer layer covalently bonded to the functional layer, or covalently bonded via a spacer system, to the functional polymer layer.

10. The method according to claim 9, wherein the covalently bonded polymer is a thermosensitive polymer which at a temperature below 36° C. in the active ingredient-containing medium has an open structure into which active ingredient molecules can be incorporated and at temperatures ≧36° C. has a closed structure in which the active ingredient molecules are enclosed.

11. The method according to claim 10, wherein the the active ingredient is tretinoin, tretinoin derivatives, orphan receptor agonists, elafin derivatives, corticosteroids, steroid hormones, taxol, taxol derivatives, rapamune, tacrolimus, hydrophobic proteins or cell proliferation-altering substances.

12. The method according to claim 1, wherein the functional polymer layer deposited on the endoprosthesis has a layer thickness between 200-400 nm.

13. The method according to claim 1, wherein the functional polymer is polyamine-p-xylylene-co-polyxylylene.

14. The method according to claim 4, wherein the active ingredient is water insoluble.

15. A method of producing bioactive surfaces on an endoprosthesis, wherein the endoprosthesis comprises a functional polymer layer and an active ingredient-containing layer positioned on the functional polymer layer, the method comprising:
(a) depositing a first polymer of general structures (1), (2) or (3) or a combination thereof; 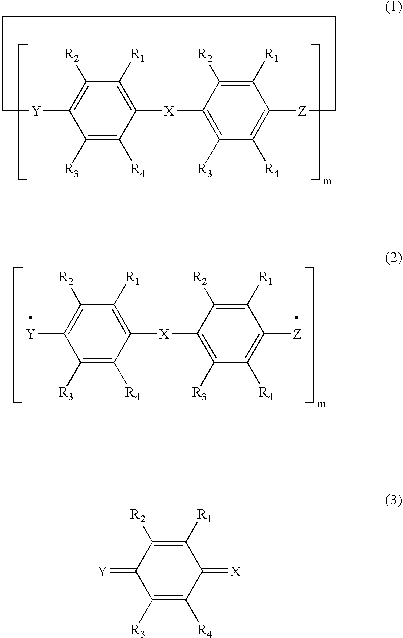
wherein R_{1,2,3,4}: are, equal or different and comprising, hydrogen atoms, halogen atoms, alkyl groups, substituted alkyl groups, aryl groups, substituted aryl groups, organic residues, organic radicals, a compound of the general structure CO (O-M-A), -wherein M: is an aliphatic or aromatic groups;
A is a hydrogen, hydroxyl group, amino group, carboxyl group, metallated group, hydroxyl group, amino group, carboxyl group, ester group, ether group, acid halide group, isocyanate group, sulfur containing group, nitrogen containing group, phosphorus containing group, or silicon containing group,
X, Y is a hydrocarbon residues
m: number of repeating units=1-20,
at temperatures from about 500 to about 1000° C. and pressures less than 500 Pa to generate monomers in a gas phase;
(b) reducing the temperature to polymerize the functional polymer layer and form a polymer-coated endoprosthesis;
(c) wetting the polymer-coated endoprosthesis in a water misicible solvent to form a wetted polymer-coated endoprosthesis;
(d) immersing the wetted polymer-coated endoprosthesis in water; and
(e) adding the active ingredient to the water, wherein the active ingredient is poorly soluble in water and precipitates to at least partially deposits on functional polymer layer.

16. The method according to claim 15, wherein the wetting agent is dimethylsulfoxide (DMSO), dioxane, dimethylformamide (DMF) or tetrahydrofuran (THF).

17. The method according to claim 15, wherein the active ingredient is tretinoin, tretinoin derivatives, orphan receptor agonists, elafin derivatives, corticosteroids, steroid hormones, taxol, taxol derivatives, rapamune, or tacrolimus.

18. The method according to claim 15, wherein the active ingredient layer is a second polymer layer covalently bonded to the functional layer, wherein the second polymer layer comprises a thermosensitive polymer which at a temperature below 36° C. in the active ingredient-containing medium has an open structure into which active ingredient molecules can be incorporated and at temperatures ≧36° C. has a closed structure in which the active ingredient molecules are enclosed.

19. The method according to claim 18, wherein the thermosensitive polymer is a hydrogel.

20. The method according to claim 19, wherein the first polymer is a dimmer 4-amino-[2,2]-paracyclophane.
